(19) United States
(12) Patent Application Publication
(10) Pub. No.: US 2020/0108030 A1
(43) Pub. Date: Apr. 9, 2020

(54) COMPOSITIONS OF ALBUTEROL AND HYPERTONIC SALINE AND USES THEREOF

(71) Applicant: Imtiaz Khurshid, Ashburn, VA (US)

(72) Inventor: Imtiaz Khurshid, Ashburn, VA (US)

(21) Appl. No.: 16/593,467

(22) Filed: Oct. 4, 2019

Related U.S. Application Data
(60) Provisional application No. 62/741,963, filed on Oct. 5, 2018.

(51) Int. Cl. A61K 31/137; A61K 47/02; A61K 9/00

(52) U.S. Cl. CPC A61K 31/137; A61K 9/0073; A61K 9/0043; A61K 47/02

(57) ABSTRACT

Provided herein are pharmaceutical compositions comprising a therapeutically effective amount of albuterol and a therapeutically effective amount of hypertonic saline. Also provided here are methods of using the aforementioned pharmaceutical compositions to treat one or more symptoms of obstructive airway diseases.

### CROSS-REFERENCE TO RELATED APPLICATIONS

This application claims priority to U.S. Provisional Patent Application Ser. No. 62/741,963, filed Oct. 5, 2018; the entire contents of which are herein incorporated by reference.

### TECHNICAL FIELD

This disclosure relates to compositions comprising albuterol and hypertonic saline, methods of formulating such compositions for efficient inhalation or intranasal delivery, and methods of using such compositions to facilitate mucus clearance in obstructive airway diseases.

### BACKGROUND

Obstructive airway diseases are characterized by decreased clearance of mucus from bronchial airway surfaces and increased likelihood of pathogens (e.g., virus, bacterium, prion, fungus, viroid, or parasite) colonizing bronchial airway surfaces and triggering host immune and inflammatory responses. Representative obstructive airway diseases include, but are not limited to, chronic bronchitis, cystic fibrosis, bronchiectasis, and chronic obstructive pulmonary disease.

Inhaled selective β2-agonists (e.g. albuterol) are known to effectively treat one or more symptoms of obstructive airway diseases (Cazolla et al., Am. J. Respir. Crit. Care Med. 187:690-696, 2013); Gabriella et al., Current Opin. Pharmacol. 40:142-146, 2018). Selective β2-agonists stimulate β2-adrenergic receptors in the lungs and in doing so, indirectly trigger activation of adenylate cyclase, an enzyme that catalyzes the conversion of ATP to cyclic-3′, 5′-adenosine monophosphate (cAMP). The increased concentrations of cAMP confer relaxation of bronchial smooth muscle and reduction of inflammatory cell mediator release (Johnson, J. Allergy Clin. Immunol. 117(1):18-24, 2006). Common side effects of inhaled albuterol include, but are not limited to, headache, dizziness, insomnia, cough, hoarseness, sore throat, nausea, vomiting, dry mouth, muscle pain, diarrhea, bronchospasm (e.g. wheezing, chest tightness, and trouble breathing), decreased potassium, and increased blood pressure (see, e.g., rxlist.com and webmd.com websites).

### SUMMARY

The present invention is based on the discovery that co-administration of albuterol and hypertonic saline in a single pharmaceutical composition enhances the therapeutic effect of hypertonic saline while mitigating one or more side effects of hypertonic saline. Based on this discovery provided herein are pharmaceutical compositions comprising a therapeutically effective amount of albuterol and a therapeutically effective amount of hypertonic saline. Also provided herein are methods of preparing the aforementioned pharmaceutical compositions either for administration by inhalation or intranasal. Also provided herein are methods of treating symptoms of obstructive airway diseases in a subject in need thereof that include administering a therapeutically effective amount of the aforementioned pharmaceutical compositions to the subject. Exemplary obstructive airway diseases include, but are not limited to, chronic bronchitis, cystic fibrosis, bronchiectasis, and chronic obstructive pulmonary disease.

In some embodiments, administration of any of the pharmaceutical compositions described herein can increase patient compliance and reduce medical cost by eliminating the need for separate use (and separate administration) of the albuterol and hypertonic saline.

As used herein, the term “albuterol” comprises forms selected from the following group: tautomeric forms, enantiomeric forms, stereoisomers, acid addition salts, base salts, solvates, analogues and derivatives of albuterol, and any mixtures thereof. In some embodiments, albuterol salts are utilized. The chemical structure of albuterol and acceptable salts of albuterol are described in U.S. Pat. Nos. 3,644,353 and 8,084,461. Non-limiting sources of albuterol are described herein.

As used herein, the term “hypertonic saline” refers to compositions comprising water and sodium chloride (NaCl) with a concentration of sodium chloride higher than physiologic concentration (0.9% w/v). In some embodiments, sodium chloride concentration is 0.9% w/v to 1.5% w/v, 1.5% w/v to 2.0% w/v, 2.0% w/v to 2.5% w/v, 2.5% w/v to 3.0% w/v, 3.0% w/v to 3.5% w/v, 3.5% w/v to 4.0% w/v, 4.0% w/v to 4.5% w/v, 4.5% w/v to 5.0% w/v, 5.0% w/v to 5.5% w/v, 5.5% w/v to 6.0% w/v, 6.0% w/v to 6.5% w/v, 6.5% w/v to 7.0% w/v, 7.0% w/v to 7.5% w/v, 7.5% w/v to 8.0% w/v, 8.0% w/v to 8.5% w/v, 8.5% w/v to 9.0% w/v, 9.0% w/v to 9.5% w/v, or 9.5% w/v to 10.0% w/v.

The term “treatment” means to ameliorate to reduce the number, frequency, and/or severity of one or more symptoms of a disorder in a subject (e.g., a human). In some examples, the disorder being treated is a mucus-producing obstructive airway disease and one or more symptoms of the obstructive airway disease includes increasing clearance from bronchial airway surfaces, decreasing likelihood of pathogen colonization of bronchial airway surfaces, decreasing host immune and inflammatory responses in bronchial airways, or increasing hydration of bronchial airway surfaces. Generally, the methods of treatment include administering a therapeutically effective amount of any of the compositions provided herein to a subject who is in need of, or who has been determined to be in need of, such treatment.

The term “subject” refers to any mammal. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine, or primate animals) may be employed.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION

Provided herein are pharmaceutical compositions that include a therapeutically effective amount of albuterol and a therapeutically effective amount of hypertonic saline. Also provided herein are methods of treating symptoms of cystic fibrosis, bronchiectasis, chronic bronchitis and other obstructive airway diseases in a subject that include administering a therapeutically effective amount of any of these pharmaceutical compositions to the subject. Also provided herein are methods of enhancing mucociliary clearance and mucosal hydration in a subject in need thereof that include administering a therapeutically effective amount of any of these pharmaceutical compositions to the subject.

### Albuterol

As used herein, the term “albuterol” includes, but is not limited to, any form of albuterol that is capable of producing a desired bronchodilation effect in patients, including, but not limited to, all tautomeric forms, enantiomeric forms, stereoisomers, anhydrides, acid addition salts, base salts, solvates, analogues and derivatives of albuterol, or any mixture thereof. In the present invention, acceptable salts of albuterol may include, but are not limited to, hydrochloride, sulfate, maleate, tartrate, citrate and the like. Additional exemplary salts of albuterol are described in U.S. Pat. No. 3,644,353, which is incorporated herein by reference in its entirety. In the present invention, the salt of albuterol can be sulfate. In an alternative embodiment, the present compositions include the sulfate salt of racemic albuterol, or it may include at least substantially of a single isomer of albuterol. Albuterol sulfate is a relatively selective beta-2-adrenergic bronchodilator with an empirical formula of C₁₃H₂₁NO₃. The chemical name for albuterol sulfate is α¹-[(tert-butylamino)methyl]-4-hydroxy-m-xylene-α,α′-diol sulfate (2:1) (salt), and its established chemical structure is as follows:

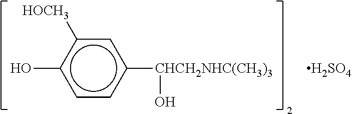

TABLE 1
Commercial sources of albuterol (PubChem Open Chemistry Database
Compound Summary for CID 0884233; Accessed Sep. 5, 2018).

| Vendor Name | Vendor Code |
|---|---|
| ABBLIS Chemicals | AB1009413 |
| Selleckchem | S2507 |
| Chem-Space.com Database | CSC020639000 |
| Chembase.cn | 73052 |
| Adooq BioScience | Salbutamol sulfate - Albuterol |
| BioChemPartner | BCP02974, BCP0726000244 |
| OXCHEM CORPORATION | AX8292143 |
| Boc Sciences | 51022-70-9 |
| Hangzhou APIChem Technology | AC-6369 |
| King Scientific | KSC593K0D |
| AK Scientific, Inc. (AKSCI) | 2200AH |
| NIH Clinical Collection | SAM001246971 |
| TargetMol | T6652 |
| Wubei-Biochem | WB463373ST |
| Angene Chemical | AGN-PC-088NIS |
| Glentham Life Sciences Ltd. | GP1722 |
| Alfa Chemistry | 51022-70-9 |
| BLD Pharm | BD150752, BD286870 |
| AKos Consulting & Solutions | AKOS015951296 |
| Molepedia | M90257121P |
| Aurora Fine Chemicals LLC | A24.076.286 |
| Chemieliva Pharmaceutical Co., Ltd | PBCM0551127 |
| Uchem Meditech | U3695 |
| LabNetwork, a WuXi AppTec Company | LN00224692 |
| ChemTik | CTK4J3501 |
| MolPort | MolPort-005-933-644 |
| Ambinter | Amb17607767 |

### Hypertonic Saline

The present invention relies on the co-formulation (and co-administration) of hypertonic saline with albuterol to reduce (e.g., a 1% to 99% reduction, a 1% to 95% reduction, a 1% to 90% reduction, a 1% to 85% reduction, a 1% to 80% reduction, a 1% to 75% reduction, a 1% to 70% reduction, a 1% to 65% reduction, a 1% to 60% reduction, a 1% to 55% reduction, a 1% to 50% reduction, a 1% to 45% reduction, a 1% to 40% reduction, a 1% to 35% reduction, a 1% to 30% reduction, a 1% to 25% reduction, a 1% to 20% reduction, a 1% to 15% reduction, a 1% to 10% reduction, a 1% to 5% reduction, a 5% to 99% reduction, a 5% to 95% reduction, a 5% to 90% reduction, a 5% to 85% reduction, a 5% to 80% reduction, a 5% to 75% reduction, a 5% to 70% reduction, a 5% to 65% reduction, a 5% to 60% reduction, a 5% to 55% reduction, a 5% to 50% reduction, a 5% to 45% reduction, a 5% to 40% reduction, a 5% to 35% reduction, a 5% to 30% reduction, a 5% to 25% reduction, a 5% to 20% reduction, a 5% to 15% reduction, a 5% to 10% reduction, a 10% to 99% reduction, a 10% to 95% reduction, a 10% to 90% reduction, a 10% to 85% reduction, a 10% to 80% reduction, a 10% to 75% reduction, a 10% to 70% reduction, a 10% to 65% reduction, a 10% to 60% reduction, a 10% to 55% reduction, a 10% to 50% reduction, a 10% to 45% reduction, a 10% to 40% reduction, a 10% to 35% reduction, a 10% to 30% reduction, a 10% to 25% reduction, a 10% to 20% reduction, a 10% to 15% reduction, a 15% to 99% reduction, a 15% to 95% reduction, a 15% to 90% reduction, a 15% to 85% reduction, a 15% to 80% reduction, a 15% to 75% reduction, a 15% to 70% reduction, a 15% to 65% reduction, a 15% to 60% reduction, a 15% to 55% reduction, a 15% to 50% reduction, a 15% to 45% reduction, a 15% to 40% reduction, a 15% to 35% reduction, a 15% to 30% reduction, a 15% to 25% reduction, a 15% to 20% reduction, a 20% to 99% reduction, a 20% to 95% reduction, a 20% to 90% reduction, a 20% to 85% reduction, a 20% to 80% reduction, a 20% to 75% reduction, a 20% to 70% reduction, a 20% to 65% reduction, a 20% to 60% reduction, a 20% to 55% reduction, a 20% to 50% reduction, a 20% to 45% reduction, a 20% to 40% reduction, a 20% to 35% reduction, a 20% to 30% reduction, a 20% to 25% reduction, a 25% to 99% reduction, a 25% to 95% reduction, a 25% to 90% reduction, a 25% to 85% reduction, a 25% to 80% reduction, a 25% to 75% reduction, a 25% to 70% reduction, a 25% to 65% reduction, a 25% to 60% reduction, a 25% to 55% reduction, a 25% to 50% reduction, a 25% to 45% reduction, a 25% to 40% reduction, a 25% to 35% reduction, a 25% to 30% reduction, a 30% to 99% reduction, a 30% to 95% reduction, a 30% to 90% reduction, a 30% to 85% reduction, a 30% to 80% reduction, a 30% to 75% reduction, a 30% to 70% reduction, a 30% to 65% reduction, a 30% to 60% reduction, a 30% to 55% reduction, a 30% to 50% reduction, a 30% to 45% reduction, a 30% to 40% reduction, a 30% to 35% reduction, a 35% to 99% reduction, a 35% to 95% reduction, a 35% to 90% reduction, a 35% to 85% reduction, a 35% to 80% reduction, a 35% to 75% reduction, a 35% to 70% reduction, a 35% to 65% reduction, a 35% to 60% reduction, a 35% to 55% reduction, a 35% to 50% reduction, a 35% to 45% reduction, a 35% to 40% reduction, a 40% to 99% reduction, a 40% to 95% reduction, a 40% to 90% reduction, a 40% to 85% reduction, a 40% to 80% reduction, a 40% to 75% reduction, a 40% to 70% reduction, a 40% to 65% reduction, a 40% to 60% reduction, a 40% to 55% reduction, a 40% to 50% reduction, a 40% to 45% reduction, a 45% to 99% reduction, a 45% to 95% reduction, a 45% to 90% reduction, a 45% to 85% reduction, a 45% to 80% reduction, a 45% to 75% reduction, a 45% to 70% reduction, a 45% to 65% reduction, a 45% to 60% reduction, a 45% to 55% reduction, a 45% to 50% reduction, a 50% to 99% reduction, a 50% to 95% reduction, a 50% to 90% reduction, a 50% to 85% reduction, a 50% to 80% reduction, a 50% to 75% reduction, a 50% to 70% reduction, a 50% to 65% reduction, a 50% to 60% reduction, a 50% to 55% reduction, a 55% to 99% reduction, a 55% to 95% reduction, a 55% to 90% reduction, a 55% to 85% reduction, a 55% to 80% reduction, a 55% to 75% reduction, a 55% to 70% reduction, a 55% to 65% reduction, a 55% to 60% reduction, a 60% to 99% reduction, a 60% to 95% reduction, a 60% to 90% reduction, a 60% to 85% reduction, a 60% to 80% reduction, a 60% to 75% reduction, a 60% to 70% reduction, a 60% to 65% reduction, a 65% to 99% reduction, a 65% to 95% reduction, a 65% to 90% reduction, a 65% to 85% reduction, a 65% to 80% reduction, a 65% to 75% reduction, a 65% to 70% reduction, a 70% to 99% reduction, a 70% to 95% reduction, a 70% to 90% reduction, a 70% to 85% reduction, a 70% to 80% reduction, a 70% to 75% reduction, a 75% to 99% reduction, a 75% to 95% reduction, a 75% to 90% reduction, a 75% to 85% reduction, a 75% to 80% reduction, a 80% to 99% reduction, a 80% to 95% reduction, a 80% to 90% reduction, a 80% to 85% reduction, a 85% to 99% reduction, a 85% to 95% reduction, a 85% to 90% reduction, a 90% to 99% reduction, a 90% to 95% reduction, or a 95% to 99% reduction) the number, severity, and/or frequency of one or more side effects associated with administration of albuterol (e.g., any of the exemplary side effects associated with administration of albuterol described herein or known in the art), e.g., as compared to the number, severity, and/or frequency of the one or more side effects in a subject administered albuterol alone.

The present invention relies on the co-formulation (and co-administration) of hypertonic saline with albuterol to reduce (e.g., a 1% to 99% reduction, or any of the subranges of this range described herein) the number, severity, and/or frequency of one or more side effects associated with administration of hypertonic saline (e.g., any of the exemplary side effects associated with administration of hypertonic saline described herein or known in the art), e.g., as compared to the number, severity, and/or frequency of the one or more side effects in a subject administered hypertonic saline alone. Non-limiting side effects of hypertonic saline include coughing and/or chest tightness.

As used herein, the term “hypertonic saline” refers to compositions comprising water and sodium chloride (NaCl) with a concentration of sodium chloride higher than physiologic concentration (0.9% w/v).

TABLE 2
Commercial sources of albuterol (PubChem Open Chemistry Database
Compound Summary for CID 5234; Accessed Sep. 5, 2018).

| Vendor Name | Vendor Code |
|---|---|
| Fisher Chemical | S2711; S27110; |
| | S2713; S271350LB; S27150; S271500; S64010; S6403; |
| | S640350LB; S64050; S640500; S641212; S641350LB; |
| | S641500; S641P350LB; S64212; S642212; S642350LB; |
| | S642500; S67110; S6713; S671500; SB17520; |
| | SB1754; SS139100; SS139500; |
| Sigma Aldrich | 02589_SIAL; 03370_SIAL; 05- |
| | 2745_SAJ; 07982_SIAL; 13423_SIGALD; 1613804_USP; |
| | 16818_SIGMA; 204439_ALDRICH; 223514_ALDRICH; |
| | 28-2260_SAJ; 28-2270_SAJ; 28- |
| | 2280_SAJ; 28-2300_SAJ; 28-2320_SAJ; 28- |
| | 4150_SAJ; 310166_SIAL; 31434_SIGALD; 378860_ALDRICH; |
| | 433209_ALDRICH; 450006_ALDRICH; |
| | 450014_ALDRICH; 52455_SIGMA; 59222C_SIGMA; |
| | 71177_SIAL; 71376_SIGMA; 71380_SIGALD; |
| | 71382_SIGALD; 71383_SIAL; 71386_SIGMA; |
| | 71387_SIAL; 71392_SIGALD; 746398_SIGALD; |
| | 793566_SIGALD; NIST2201_SIAL; NIST919B_SIAL; |
| | NIST975A_SIAL; PHR1321_SIAL; RES0926S- |
| | A7_SIGMA; S0817_SIAL; S1679_SIAL; S3014_SIGMA; |
| | S5150_SIGMA; S5886_SIGMA; S6150_SIAL; |
| | S6191_SIGMA; S6546_SIAL; S7653_SIAL; S8776_SIGMA; |
| | S9625_SIGALD; S9888_SIGALD; V000106_VETEC; |
| | V001230_VETEC; V001474_VETEC; |
| | V001542_VETEC; V003132_VETEC; V0T0013_VETEC; |
| | V0T0205_VETEC; V800372_VETEC; |
| | V800373_VETEC; V900058_VETEC; |
| abcr GmbH | AB118689; AB121999; AB122001; AB152619; AB202258; |
| | AB202971; AB203375; AB205027; AB207657; |
| | AB207745; AB208335; AB210343; AB211420; |
| | AB252754; AB252755; AB252756; AB252762; AB252763; |
| | AB252765; AB252772; AB252773 ; AB252774; |
| | AB252775; AB252782; AB252783; AB252800; |
| | AB252801; AB252803; AB252828; AB252833; |
| | AB355001; AB355002; AB355003 |
| Oakwood Products | 098526; 098804 |
| NORRIS PHARM | TH-MA10321 |
| AKos Consulting & Solutions | AKOS024438089; AKOS024457457 |
| Molepedia | M90109456P |
| Chembase.cn | 102739 |
| MP Biomedicals | 102892; 152575; 194738; 194848 |
| Finetech Industry Limited | FT-0645114 |
| AA BLOCKS | AA0035IN |
| Changzhou Highassay | my_sub3372 |
| Chemical Co., Ltd | |
| Phion Ltd | 46722343 |
| Tractus | RTR-032980; TR-032980; TRA-0176326 |
| 3B Scientific (Wuhan) Corp | 3B4-1967 |
| MuseChem | M057929; R044565 |
| Angene Chemical | AGN-PC-0U9TFM |
| Hairui Chemical | HR133170 |
| Alfa Chemistry | 7647-14-5 |
| MolPort | MolPort-003-925-236; MolPort-003-933-040 |
| AN PharmaTech | AN-21810 |
| Boc Sciences | 7647-14-5 |
| Ambinter | Amb22052533 |
| King Scientific | KS-00000VGU; KSC377C6D |
| ApexBio Technology | B7288 |
| Chemieliva Pharmaceutical | PBCM0820478 |
| Co., Ltd | |
| CambridgeChem | T81793 |
| Tocris Bioscience | 3146 |
| Combi-Blocks | QI-4971 |
| ChemTik | CTK2H7161 |
| Parchem | 919 |
| Aurora Fine Chemicals LLC | A17.875.814 |
| Glentham Life Sciences Ltd. | GE8374 |
| Wubei-Biochem | WB443981ST |
| VladaChem | VL144274 |
| AK Scientific, Inc. (AKSCI) | Z3733 |
| Achemica | ACMC-20ajon |
| Nanjing Kaimubo | KB-60515 |
| TCI (Tokyo Chemical Industry) | S0572 |

### Pharmaceutical Compositions

In the present invention, the albuterol and hypertonic saline may be provided in a variety of pharmaceutically acceptable vehicles, including, but not limited to water or any other aqueous solution comprising a pharmaceutically acceptable amount of osmotic agent.

As used herein, the phrase “therapeutically effective amount” means a safe and tolerable amount, as based on industry and/or regulatory standards. Such amount can be, e.g., sufficient to induce bronchodilation, enhance mucociliary clearance, and/or reducing immune and inflammatory responses, in lungs of patients suffering from a obstructive airway disease. Such amount can be, e.g., sufficient to enhance the therapeutic effects of hypertonic saline, while reducing one or more negative side effects of hypertonic saline that are apparent in subjects administered hypertonic saline without albuterol.

In the pharmaceutical compositions, a therapeutically effective amount of albuterol can be about 2.5 mg albuterol. In another alternative embodiment, a therapeutically effective amount of albuterol can be 0.5 mg to 1.0 mg, 1.0 mg to 1.5 mg, 1.5 mg to 2.0 mg, 2.0 mg to 2.5 mg, 2.5 mg to 3.0 mg, 3.0 mg to 3.5 mg, 3.5 mg to 4.0 mg, 4.0 mg to 4.5 mg, or 4.5 mg to 5.0 mg. In another embodiment of the present invention, a therapeutically effective amount of albuterol may include from about 0.02% w/v to about 5.0% w/v albuterol, including the following intermediate ranges: 0.02% w/v to 0.05% w/v, 0.05% w/v to 1.0% w/v, 1.0% w/v to 1.5% w/v, 1.5% w/v to 2.0% w/v, 2.0% w/v to 2.5% w/v, 2.5% w/v to 3.0% w/v, 3.0% w/v to 3.5% w/v, 3.5% w/v to 4.0% w/v, 4.0% w/v to 4.5% w/v, or 4.5% w/v to 5.0% w/v.

Any of the compositions provided herein can be stable for 30 days at 25° C. In some examples, any of the compositions provided herein can have greater than 80%, 85%, 90%, or 95% of the initial amount of active ingredients present in the composition at 30 days following storage at 25° C.

In some embodiments, the pharmaceutical composition may have a pH of about 2.0 to about 8.0. In some embodiments, the compositions may have a pH of about 3.0 to about 4.0, or a pH of about 3.5. The pH may be adjusted with hydrochloric acid or sulfuric acid. Exemplary buffer components include, but are not limited to, acetate, citrate, formate, maleate, phosphate, succinate, BIS-TRIS, and HEPES.

In some embodiments, the osmolality of any of the pharmaceutical compositions provided herein may be adjusted from about 150 to about 550 mOsm/kg by addition of a tonicity adjusting agent. Exemplary tonicity adjusting agents include, but are not limited to, ammonium carbonate, ammonium chloride, ammonium lactate, ammonium nitrate, ammonium phosphate, ammonium sulfate, ascorbic acid, bismuth, sodium tartrate, boric acid, calcium chloride, calcium, disodium edetate, calcium gluconate, calcium lactate, citric acid, dextrose, diethanolamine, dimethyl sulfoxide, edetate disodium, edetate trisodium monohydrate, fluorescein, sodium, fructose, galactose, glycerin, lactic acid, lactose, magnesium chloride, magnesium sulfate, manitol, polyethyne glycol, potassium acetate, potassium chlorate, potassium chloride, potassium iodide, potassium nitrate, potassium phosphate, potassium sulfate, propylene glycol, silver nitrate, sodium acetate, sodium bicarbonate, sodium biphosphate, sodium bisulfate, sodium borate, sodium bromide, sodium cacodylate, sodium carbonate, sodium chloride, sodium citrate, sodium iodide, sodium lactate, sodium metabisulfite, sodium nitrate, sodium nitrite, sodium phosphate, sodium propionate, sodium succinate, sodium sulfate, sodium sulfite, sodium tartrate, sodium thiosulfate, sorbitol, sucrose, tartaric acid, triethanolamine, urea, urethan, uridine, and zinc sulfate.

In some embodiments, the pharmaceutical compositions are formulated for different routes of administration (e.g., inhalation or intranasal). In some embodiments, the pharmaceutical compositions are provided in sterile, unit dose treatments. Single or multiple administrations of pharmaceutical compositions can be given to a subject in need thereof depending on for example: the dosage and frequency as required and tolerated by the subject. The formulation should provide a sufficient quantity of each active agent (e.g., albuterol and hypertonic saline) to effectively treat, prevent, or ameliorate conditions, diseases, or symptoms. In some embodiments, the pharmaceutical composition can be provided as a sterile, nebulizer solution comprising about 0.75 mg/3 mL to 3.0 mg/3 mL of albuterol (e.g., albuterol sulfate).

### Methods of Treatment

Also provided herein are methods of treating a subject in need thereof (e.g., any of the exemplary subjects described herein or known in the art) that include administering to the subject a therapeutically effective amount of any of the pharmaceutical compositions described herein.

In some embodiments of these methods, the subject has been identified or diagnosed as having an obstructive airway disease. Non-limiting examples of obstructive airway diseases include, but are not limited to, chronic bronchitis, cystic fibrosis, bronchiectasis, and chronic obstructive pulmonary disease.

In some embodiments, these methods can result in a reduction in the number, severity, or frequency of one or more symptoms of the obstructive airway disease in the subject (e.g., as compared to the number, severity, or frequency of the one or more symptoms of the obstructive airway disease in the subject prior to treatment). In some embodiments, these methods can result in a reduction (e.g., about 1% reduction to about 99% reduction, about 1% reduction to about 95% reduction, about 1% reduction to about 90% reduction, about 1% reduction to about 85% reduction, about 1% reduction to about 80% reduction, about 1% reduction to about 75% reduction, about 1% reduction to about 70% reduction, about 1% reduction to about 65% reduction, about 1% reduction to about 60% reduction, about 1% reduction to about 55% reduction, about 1% reduction to about 50% reduction, about 1% reduction to about 45% reduction, about 1% reduction to about 40% reduction, about 1% reduction to about 35% reduction, about 1% reduction to about 30% reduction, about 1% reduction to about 25% reduction, about 1% reduction to about 20% reduction, about 1% reduction to about 15% reduction, about 1% reduction to about 10% reduction, about 1% reduction to about 5% reduction, about 5% reduction to about 99% reduction, about 5% reduction to about 95% reduction, about 5% reduction to about 90% reduction, about 5% reduction to about 85% reduction, about 5% reduction to about 80% reduction, about 5% reduction to about 75% reduction, about 5% reduction to about 70% reduction, about 5% reduction to about 65% reduction, about 5% reduction to about 60% reduction, about 5% reduction to about 55% reduction, about 5% reduction to about 50% reduction, about 5% reduction to about 45% reduction, about 5% reduction to about 40% reduction, about 5% reduction to about 35% reduction, about 5% reduction to about 30% reduction, about 5% reduction to about 25% reduction, about 5% reduction to about 20% reduction, about 5% reduction to about 15% reduction, about 5% reduction to about 10% reduction, about 10% reduction to about 99% reduction, about 10% reduction to about 95% reduction, about 10% reduction to about 90% reduction, about 10% reduction to about 85% reduction, about 10% reduction to about 80% reduction, about 10% reduction to about 75% reduction, about 10% reduction to about 70% reduction, about 10% reduction to about 65% reduction, about 10% reduction to about 60% reduction, about 10% reduction to about 55% reduction, about 10% reduction to about 50% reduction, about 10% reduction to about 45% reduction, about 10% reduction to about 40% reduction, about 10% reduction to about 35% reduction, about 10% reduction to about 30% reduction, about 10% reduction to about 25% reduction, about 10% reduction to about 20% reduction, about 10% reduction to about 15% reduction, about 15% reduction to about 99% reduction, about 15% reduction to about 95% reduction, about 15% reduction to about 90% reduction, about 15% reduction to about 85% reduction, about 15% reduction to about 80% reduction, about 15% reduction to about 75% reduction, about 15% reduction to about 70% reduction, about 15% reduction to about 65% reduction, about 15% reduction to about 60% reduction, about 15% reduction to about 55% reduction, about 15% reduction to about 50% reduction, about 15% reduction to about 45% reduction, about 15% reduction to about 40% reduction, about 15% reduction to about 35% reduction, about 15% reduction to about 30% reduction, about 15% reduction to about 25% reduction, about 15% reduction to about 20% reduction, about 20% reduction to about 99% reduction, about 20% reduction to about 95% reduction, about 20% reduction to about 90% reduction, about 20% reduction to about 85% reduction, about 20% reduction to about 80% reduction, about 20% reduction to about 75% reduction, about 20% reduction to about 70% reduction, about 20% reduction to about 65% reduction, about 20% reduction to about 60% reduction, about 20% reduction to about 55% reduction, about 20% reduction to about 50% reduction, about 20% reduction to about 45% reduction, about 20% reduction to about 40% reduction, about 20% reduction to about 35% reduction, about 20% reduction to about 30% reduction, about 20% reduction to about 25% reduction, about 25% reduction to about 99% reduction, about 25% reduction to about 95% reduction, about 25% reduction to about 90% reduction, about 25% reduction to about 85% reduction, about 25% reduction to about 80% reduction, about 25% reduction to about 75% reduction, about 25% reduction to about 70% reduction, about 25% reduction to about 65% reduction, about 25% reduction to about 60% reduction, about 25% reduction to about 55% reduction, about 25% reduction to about 50% reduction, about 25% reduction to about 45% reduction, about 25% reduction to about 40% reduction, about 25% reduction to about 35% reduction, about 25% reduction to about 30% reduction, about 30% reduction to about 99% reduction, about 30% reduction to about 95% reduction, about 30% reduction to about 90% reduction, about 30% reduction to about 85% reduction, about 30% reduction to about 80% reduction, about 30% reduction to about 75% reduction, about 30% reduction to about 70% reduction, about 30% reduction to about 65% reduction, about 30% reduction to about 60% reduction, about 30% reduction to about 55% reduction, about 30% reduction to about 50% reduction, about 30% reduction to about 45% reduction, about 30% reduction to about 40% reduction, about 30% reduction to about 35% reduction, about 35% reduction to about 99% reduction, about 35% reduction to about 95% reduction, about 35% reduction to about 90% reduction, about 35% reduction to about 85% reduction, about 35% reduction to about 80% reduction, about 35% reduction to about 75% reduction, about 35% reduction to about 70% reduction, about 35% reduction to about 65% reduction, about 35% reduction to about 60% reduction, about 35% reduction to about 55% reduction, about 35% reduction to about 50% reduction, about 35% reduction to about 45% reduction, about 35% reduction to about 40% reduction, about 40% reduction to about 99% reduction, about 40% reduction to about 95% reduction, about 40% reduction to about 90% reduction, about 40% reduction to about 85% reduction, about 40% reduction to about 80% reduction, about 40% reduction to about 75% reduction, about 40% reduction to about 70% reduction, about 40% reduction to about 65% reduction, about 40% reduction to about 60% reduction, about 40% reduction to about 55% reduction, about 40% reduction to about 50% reduction, about 40% reduction to about 45% reduction, about 45% reduction to about 99% reduction, about 45% reduction to about 95% reduction, about 45% reduction to about 90% reduction, about 45% reduction to about 85% reduction, about 45% reduction to about 80% reduction, about 45% reduction to about 75% reduction, about 45% reduction to about 70% reduction, about 45% reduction to about 65% reduction, about 45% reduction to about 60% reduction, about 45% reduction to about 55% reduction, about 45% reduction to about 50% reduction, about 50% reduction to about 99% reduction, about 50% reduction to about 95% reduction, about 50% reduction to about 90% reduction, about 50% reduction to about 85% reduction, about 50% reduction to about 80% reduction, about 50% reduction to about 75% reduction, about 50% reduction to about 70% reduction, about 50% reduction to about 65% reduction, about 50% reduction to about 60% reduction, about 50% reduction to about 55% reduction, about 55% reduction to about 99% reduction, about 55% reduction to about 95% reduction, about 55% reduction to about 90% reduction, about 55% reduction to about 85% reduction, about 55% reduction to about 80% reduction, about 55% reduction to about 75% reduction, about 55% reduction to about 70% reduction, about 55% reduction to about 65% reduction, about 55% reduction to about 60% reduction, about 60% reduction to about 99% reduction, about 60% reduction to about 95% reduction, about 60% reduction to about 90% reduction, about 60% reduction to about 85% reduction, about 60% reduction to about 80% reduction, about 60% reduction to about 75% reduction, about 60% reduction to about 70% reduction, about 60% reduction to about 65% reduction, about 65% reduction to about 99% reduction, about 65% reduction to about 95% reduction, about 65% reduction to about 90% reduction, about 65% reduction to about 85% reduction, about 65% reduction to about 80% reduction, about 65% reduction to about 75% reduction, about 65% reduction to about 70% reduction, about 70% reduction to about 99% reduction, about 70% reduction to about 95% reduction, about 70% reduction to about 90% reduction, about 70% reduction to about 85% reduction, about 70% reduction to about 80% reduction, about 70% reduction to about 75% reduction, about 75% reduction to about 99% reduction, about 75% reduction to about 95% reduction, about 75% reduction to about 90% reduction, about 75% reduction to about 85% reduction, about 75% reduction to about 80% reduction, about 80% reduction to about 99% reduction, about 80% reduction to about 95% reduction, about 80% reduction to about 90% reduction, about 80% reduction to about 85% reduction, about 85% reduction to about 99% reduction, about 85% reduction to about 95% reduction, about 85% reduction to about 90% reduction, about 90% reduction to about 99% reduction, about 90% reduction to about 95% reduction, or about 95% reduction to about 99% reduction) in the number, severity, and/or frequency of one or more symptoms of the disease (e.g., any of the exemplary symptoms of obstructive airway diseases described herein) or reduction (e.g., a 1% to 99% reduction, or any of the subranges of this range described herein) in the number, severity, and/or frequency of one or more known side-effects of albuterol (e.g., any of the side effects of albuterol described herein or known in the art) in the subject, as compared to when albuterol alone at a comparable dosage is administered to similar patients or as compared to when hypertonic saline alone at a comparable dosage is administered to similar patients. Exemplary symptoms of obstructive airway diseases include reduced clearance from bronchial airway surfaces, increased likelihoods of pathogen colonization of bronchial airway surfaces, increased host immune and inflammatory responses in bronchial airways. Methods for assessing the number, frequency, and severity of symptoms of obstructive airway diseases are known in the art. Methods for assessing the number, frequency, and severity of side effects of albuterol are also known in the art.

In some embodiments, the pharmaceutical composition comprising albuterol and hypertonic saline is used to treat various clinical stages of cystic fibrosis or of chronic obstructive airway disease.

In some embodiments, these methods can result in a reduction in the number, severity, and/or frequency of one or more known side effects of hypertonic saline as compared to when hypertonic saline is administered alone (e.g., as compared to the number, severity, or frequency of one or more negative side effects of hypertonic saline observed when hypertonic saline is administered alone without albuterol) by inhalation or intranasally in similar subjects. In some embodiments, these methods can result in a reduction (e.g., about 1% reduction to about 99% reduction, about 1% reduction to about 95% reduction, about 1% reduction to about 90% reduction, about 1% reduction to about 85% reduction, about 1% reduction to about 80% reduction, about 1% reduction to about 75% reduction, about 1% reduction to about 70% reduction, about 1% reduction to about 65% reduction, about 1% reduction to about 60% reduction, about 1% reduction to about 55% reduction, about 1% reduction to about 50% reduction, about 1% reduction to about 45% reduction, about 1% reduction to about 40% reduction, about 1% reduction to about 35% reduction, about 1% reduction to about 30% reduction, about 1% reduction to about 25% reduction, about 1% reduction to about 20% reduction, about 1% reduction to about 15% reduction, about 1% reduction to about 10% reduction, about 1% reduction to about 5% reduction, about 5% reduction to about 99% reduction, about 5% reduction to about 95% reduction, about 5% reduction to about 90% reduction, about 5% reduction to about 85% reduction, about 5% reduction to about 80% reduction, about 5% reduction to about 75% reduction, about 5% reduction to about 70% reduction, about 5% reduction to about 65% reduction, about 5% reduction to about 60% reduction, about 5% reduction to about 55% reduction, about 5% reduction to about 50% reduction, about 5% reduction to about 45% reduction, about 5% reduction to about 40% reduction, about 5% reduction to about 35% reduction, about 5% reduction to about 30% reduction, about 5% reduction to about 25% reduction, about 5% reduction to about 20% reduction, about 5% reduction to about 15% reduction, about 5% reduction to about 10% reduction, about 10% reduction to about 99% reduction, about 10% reduction to about 95% reduction, about 10% reduction to about 90% reduction, about 10% reduction to about 85% reduction, about 10% reduction to about 80% reduction, about 10% reduction to about 75% reduction, about 10% reduction to about 70% reduction, about 10% reduction to about 65% reduction, about 10% reduction to about 60% reduction, about 10% reduction to about 55% reduction, about 10% reduction to about 50% reduction, about 10% reduction to about 45% reduction, about 10% reduction to about 40% reduction, about 10% reduction to about 35% reduction, about 10% reduction to about 30% reduction, about 10% reduction to about 25% reduction, about 10% reduction to about 20% reduction, about 10% reduction to about 15% reduction, about 15% reduction to about 99% reduction, about 15% reduction to about 95% reduction, about 15% reduction to about 90% reduction, about 15% reduction to about 85% reduction, about 15% reduction to about 80% reduction, about 15% reduction to about 75% reduction, about 15% reduction to about 70% reduction, about 15% reduction to about 65% reduction, about 15% reduction to about 60% reduction, about 15% reduction to about 55% reduction, about 15% reduction to about 50% reduction, about 15% reduction to about 45% reduction, about 15% reduction to about 40% reduction, about 15% reduction to about 35% reduction, about 15% reduction to about 30% reduction, about 15% reduction to about 25% reduction, about 15% reduction to about 20% reduction, about 20% reduction to about 99% reduction, about 20% reduction to about 95% reduction, about 20% reduction to about 90% reduction, about 20% reduction to about 85% reduction, about 20% reduction to about 80% reduction, about 20% reduction to about 75% reduction, about 20% reduction to about 70% reduction, about 20% reduction to about 65% reduction, about 20% reduction to about 60% reduction, about 20% reduction to about 55% reduction, about 20% reduction to about 50% reduction, about 20% reduction to about 45% reduction, about 20% reduction to about 40% reduction, about 20% reduction to about 35% reduction, about 20% reduction to about 30% reduction, about 20% reduction to about 25% reduction, about 25% reduction to about 99% reduction, about 25% reduction to about 95% reduction, about 25% reduction to about 90% reduction, about 25% reduction to about 85% reduction, about 25% reduction to about 80% reduction, about 25% reduction to about 75% reduction, about 25% reduction to about 70% reduction, about 25% reduction to about 65% reduction, about 25% reduction to about 60% reduction, about 25% reduction to about 55% reduction, about 25% reduction to about 50% reduction, about 25% reduction to about 45% reduction, about 25% reduction to about 40% reduction, about 25% reduction to about 35% reduction, about 25% reduction to about 30% reduction, about 30% reduction to about 99% reduction, about 30% reduction to about 95% reduction, about 30% reduction to about 90% reduction, about 30% reduction to about 85% reduction, about 30% reduction to about 80% reduction, about 30% reduction to about 75% reduction, about 30% reduction to about 70% reduction, about 30% reduction to about 65% reduction, about 30% reduction to about 60% reduction, about 30% reduction to about 55% reduction, about 30% reduction to about 50% reduction, about 30% reduction to about 45% reduction, about 30% reduction to about 40% reduction, about 30% reduction to about 35% reduction, about 35% reduction to about 99% reduction, about 35% reduction to about 95% reduction, about 35% reduction to about 90% reduction, about 35% reduction to about 85% reduction, about 35% reduction to about 80% reduction, about 35% reduction to about 75% reduction, about 35% reduction to about 70% reduction, about 35% reduction to about 65% reduction, about 35% reduction to about 60% reduction, about 35% reduction to about 55% reduction, about 35% reduction to about 50% reduction, about 35% reduction to about 45% reduction, about 35% reduction to about 40% reduction, about 40% reduction to about 99% reduction, about 40% reduction to about 95% reduction, about 40% reduction to about 90% reduction, about 40% reduction to about 85% reduction, about 40% reduction to about 80% reduction, about 40% reduction to about 75% reduction, about 40% reduction to about 70% reduction, about 40% reduction to about 65% reduction, about 40% reduction to about 60% reduction, about 40% reduction to about 55% reduction, about 40% reduction to about 50% reduction, about 40% reduction to about 45% reduction, about 45% reduction to about 99% reduction, about 45% reduction to about 95% reduction, about 45% reduction to about 90% reduction, about 45% reduction to about 85% reduction, about 45% reduction to about 80% reduction, about 45% reduction to about 75% reduction, about 45% reduction to about 70% reduction, about 45% reduction to about 65% reduction, about 45% reduction to about 60% reduction, about 45% reduction to about 55% reduction, about 45% reduction to about 50% reduction, about 50% reduction to about 99% reduction, about 50% reduction to about 95% reduction, about 50% reduction to about 90% reduction, about 50% reduction to about 85% reduction, about 50% reduction to about 80% reduction, about 50% reduction to about 75% reduction, about 50% reduction to about 70% reduction, about 50% reduction to about 65% reduction, about 50% reduction to about 60% reduction, about 50% reduction to about 55% reduction, about 55% reduction to about 99% reduction, about 55% reduction to about 95% reduction, about 55% reduction to about 90% reduction, about 55% reduction to about 85% reduction, about 55% reduction to about 80% reduction, about 55% reduction to about 75% reduction, about 55% reduction to about 70% reduction, about 55% reduction to about 65% reduction, about 55% reduction to about 60% reduction, about 60% reduction to about 99% reduction, about 60% reduction to about 95% reduction, about 60% reduction to about 90% reduction, about 60% reduction to about 85% reduction, about 60% reduction to about 80% reduction, about 60% reduction to about 75% reduction, about 60% reduction to about 70% reduction, about 60% reduction to about 65% reduction, about 65% reduction to about 99% reduction, about 65% reduction to about 95% reduction, about 65% reduction to about 90% reduction, about 65% reduction to about 85% reduction, about 65% reduction to about 80% reduction, about 65% reduction to about 75% reduction, about 65% reduction to about 70% reduction, about 70% reduction to about 99% reduction, about 70% reduction to about 95% reduction, about 70% reduction to about 90% reduction, about 70% reduction to about 85% reduction, about 70% reduction to about 80% reduction, about 70% reduction to about 75% reduction, about 75% reduction to about 99% reduction, about 75% reduction to about 95% reduction, about 75% reduction to about 90% reduction, about 75% reduction to about 85% reduction, about 75% reduction to about 80% reduction, about 80% reduction to about 99% reduction, about 80% reduction to about 95% reduction, about 80% reduction to about 90% reduction, about 80% reduction to about 85% reduction, about 85% reduction to about 99% reduction, about 85% reduction to about 95% reduction, about 85% reduction to about 90% reduction, about 90% reduction to about 99% reduction, about 90% reduction to about 95% reduction, or about 95% reduction to about 99% reduction) in the number, frequency, and/or severity of one or more (e.g., two, three, four, or five) known side-effects of hypertonic saline as compared to when hypertonic saline alone at comparable dosage is administered to similar subjects. Exemplary side effects of hypertonic saline include, but are not limited to, coughing and/or chest tightness.

1. A pharmaceutical composition comprising a therapeutically effective amount of albuterol and a therapeutically effective amount of hypertonic saline.

2. The pharmaceutical composition of claim 1, wherein the composition comprises an albuterol salt or solvate thereof.

3. The pharmaceutical composition of claim 1, wherein the composition further comprises a pharmaceutically acceptable carrier or vehicle.

4. The pharmaceutical composition of claim 1, wherein the composition is formulated for inhalation.

5. The pharmaceutical composition of claim 1, wherein the composition is formulation for intranasal administration.

6. A method of treating symptoms of cystic fibrosis, bronchiectasis, chronic bronchitis and other obstructive airway diseases in a subject, the method comprising administering a therapeutically effective amount of the pharmaceutical composition of claim 1 to the subject.

7. A method of enhancing mucociliary clearance and mucosal hydration in a subject in need thereof, the method comprising administering a therapeutically effective amount of pharmaceutical composition of claim 1 to the subject.

8. The method of claim 6, wherein the pharmaceutical composition is delivered as a micronized particle of respirable size.

9. The method of claim 6, wherein the pharmaceutical composition is intranasally administered to the subject.

10. The method of claim 6, wherein the pharmaceutical composition is administered to the pulmonary airways of the subject.

11. The method of claim 6, wherein the administration involves aerosolization of the pharmaceutical composition.

12. The method of claim 6, wherein the pharmaceutical composition is sterile.
